(19) United States
(12) Patent Application Publication
(10) Pub. No.: US 2009/0232796 A1
(43) Pub. Date: Sep. 17, 2009

(54) METHOD OF TREATING CANCER BY ADMINISTERING AN IMMUNOMODULATORY COMPOUND IN COMBINATION WITH A CD40 ANTIBODY OR CD40 LIGAND

(76) Inventors: Laura G. Corral, San Diego, CA (US); Helen Brady, San Diego, CA (US); Kyle Chan, San Diego, CA (US)

Correspondence Address: JONES DAY, 222 EAST 41ST ST, NEW YORK, NY, 10017, US

(21) Appl. No.: 12/390,220

(22) Filed: Feb. 20, 2009

Related U.S. Application Data
(60) Provisional application No. 61/066,578, filed on Feb. 20, 2008.

(51) Int. Cl. A61K 39/395; A61K 31/454

(52) U.S. Cl. 424/130.1; 514/323

(57) ABSTRACT

Provided herein relates to the field of cancer and its treatment by administering immunomodulatory compounds in combination with other compounds. In particular, a combination of an immunomodulatory compound and an anti-CD40 antibody and/or a CD40 ligand is provided.

This application claims priority to U.S. Provisional Application Ser. No. 61/066,578 filed Feb. 20, 2008, which is incorporated herein by reference in its entirety.

### 1. FIELD OF INVENTION

Provided herein are methods of treating cancer by administering immunomodulatory compounds in combination with other compounds.

### 2. BACKGROUND OF INVENTION

The incidence of cancer continues to climb as the general population ages and as new cancers develop. A demand exists for new and effective therapies that can be used to treat patients with cancer. Current cancer therapy may involve such methods as surgery, chemotherapy, hormonal therapy, and radiation therapy. Each of these approaches has drawbacks. Surgery, for example, may be contraindicated due to the health of a patient, and may not be effective in completely removing the cancer. Radiation therapy often elicits serious side effects. Hormonal therapy is rarely effective as a single agent.

Most treatments for hematological and solid malignancies typically aim to kill the malignant cells using cytotoxic drugs, which can have side effects such as the lulling of normal lymphocytes and impairment the patient immune response. These drugs typically act by inhibiting DNA synthesis, either directly or indirectly by inhibiting the biosynthesis of deoxyribonucleotide triphosphate precursors to prevent DNA replication and concomitant cell division (Gilman et al., 2001, Goodman and Gilman's: The Pharmacological Basis of Therapeutics, Tenth Ed.; McGraw Hill, New York). Most chemotherapeutic agents are toxic, and chemotherapy causes significant, dangerous side effects including severe nausea, bone marrow depression, lulling of normal lymphocytes, and impairment of the immune response. Therefore, a continuing need for safe and effective treatment of cancer exists.

### 3. SUMMARY OF INVENTION

Provided herein are methods and compositions for the treatment of cancer. In some embodiments, an immunomodulatory compound is administered to a patient, in combination with either a CD40 antibody, or CD40L, or both. The combination exhibits a synergistic effect that can increase the likelihood of an effective patient response.

### 4. BRIEF DESCRIPTION OF THE DRAWINGS

FIG. 1 is a bar graph illustrating that the immunomodulatory compounds 1,3-dioxo-2-(2,6-dioxopiperidin-3-yl)-4-aminoisoindoline and 1-oxo-2-(2,6-dioxopiperidin-3-yl)-4-aminoisoindoline increase the number of CD40⁺ Raji cells. The percentage of CD40⁺ cells after 48 hours of incubation with DMSO (control), 10 μM 1,3-dioxo-2-(2,6-dioxopiperidin-3-yl)-4-aminoisoindoline, or 10 μM 1-oxo-2-(2,6-dioxopiperidin-3-yl)-4-aminoisoindoline is shown.

FIG. 2 is a line graph illustrating that the immunomodulatory compounds 1,3-dioxo-2-(2,6-dioxopiperidin-3-yl)-4-aminoisoindoline and 1-oxo-2-(2,6-dioxopiperidin-3-yl)-4-aminoisoindoline increase expression of CD40 in B-CLL cells after 4 days in culture on CD5⁺ CD19⁺ CLL blasts from a newly diagnosed patient. The graph shows the mean intensity of anti-CD40 APC antibody. The mean intensity is approximately doubled by immunomodulatory compound treatment.

FIG. 3 is a bar graph illustrating the upregulation of CD40 expression in B-CLL cells by 1,3-dioxo-2-(2,6-dioxopiperidin-3-yl)-4-aminoisoindoline administration. Cells from a newly diagnosed patient were cultured with a DMSO control, or with 1,3-dioxo-2-(2,6-dioxopiperidin-3-yl)-4-aminoisoindoline at a concentration of either 0.1 μM, 1.0 μM, or 10 μM for 24 hours. The mean intensity of anti-CD40 APC antibody in CD5⁺CD19⁺ CLL blasts is shown. The results show that 1,3-dioxo-2-(2,6-dioxopiperidin-3-yl)-4-aminoisoindoline augments the expression of CD40 in a dose responsive manner.

FIG. 4 is a line graph illustrating the effect of various concentrations of 1,3-dioxo-2-(2,6-dioxopiperidin-3-yl)-4-aminoisoindoline on the proliferation of Namalwa cells, either alone (♦), with control fibroblasts (Δ), or with fibroblasts that express CD40L on the cell surface (▪). Cells were treated with either DMSO (control) or 1,3-dioxo-2-(2,6-dioxopiperidin-3-yl)-4-aminoisoindoline at either 0.01 μM, 0.1 μM, 1.0 μM, or 10 μM. The results show that 1,3-dioxo-2-(2,6-dioxopiperidin-3-yl)-4-aminoisoindoline is capable of inhibiting cell proliferation in a dose dependent manner, either alone or in the presence of control fibroblasts.

FIG. 5 is a bar graph illustrating that the administration of an immunomodulatory compound (1,3-dioxo-2-(2,6-dioxopiperidin-3-yl)-4-aminoisoindoline or 1-oxo-2-(2,6-dioxopiperidin-3-yl)-4-aminoisoindoline) increases the response of B-CLL cells to CD40L treatment. B-CLL cells from a relapsed patient at baseline were pre-treated with compounds for 48 hours. The cells were then cocultured with CD40L-transfected fibroblasts or control fibroblast for another 48 hours.

FIG. 6 is a line graph illustrating that 1-oxo-2-(2,6-dioxopiperidin-3-yl)-4-aminoisoindoline increases the response of B-CLL cells to CD40L treatment. B-CLL cells from a newly diagnosed patient at baseline were pre-treated with a DMSO control, or 1-oxo-2-(2,6-dioxopiperidin-3-yl)-4-aminoisoindoline at a concentration of either 0.1 μM, 1.0 μM, or 10 μM for 24 hours. The cells were then cultured either alone (♦), with CD40L-transfected fibroblasts (▪), or control fibroblasts (Δ) for 48 h.

FIG. 7 is a two-dimensional dot plot flow cytometry analysis demonstrating the upregulation of CD40 on CD19⁺CD5⁺ cells in a patient after 8 days of 1-oxo-2-(2,6-dioxopiperidin-3-yl)-4-aminoisoindoline therapy. At baseline, 6.4% of CD19⁺CD5⁺ cells were CD40⁺. After 8 days of therapy, CD40⁺ cells increased to 57.4%.

FIG. 8 is a two-dimensional dot plot flow cytometry analysis demonstrating the upregulation of CD40 on CD19⁺CD5⁺ cells in a patient after 8 days of 1-oxo-2-(2,6-dioxopiperidin-3-yl)-4-aminoisoindoline therapy. At baseline, 12.5% of CD19⁺CD5⁺ cells were CD40⁺. After 8 days of therapy, CD40⁺ cells increased to 36.7%.

FIG. 9 is a two-dimensional dot plot flow cytometry analysis demonstrating the upregulation of CD40 on CD19⁺CD5⁺ cells in a patient after 28 days of 1-oxo-2-(2,6-dioxopiperidin-3-yl)-4-aminoisoindoline therapy. At baseline, 2.4% of CD19⁺CD5⁺ cells were CD40⁺. After 28 days of therapy, CD40⁺ cells increased to 13.9%.

### 5. DETAILED DESCRIPTION OF THE INVENTION

Immunomodulatory compounds provided herein can be effective in treating many types of cancer. In some embodiments, administration of the immunomodulatory compounds can be combined with administration of an anti-CD40 antibody and/or CD40L.

In some embodiments, the immunomodulatory compound can be a compound known as an IMiD® immunomodulatory compound (Celgene Corporation). Exemplary immunomodulatory compounds are described herein elsewhere. As used herein and unless otherwise indicated, the term “immunomodulatory compound” can encompass certain small organic molecules that inhibit LPS induced monocyte TNF-α, IL-1β, IL-12, IL-6, MIP-1α, MCP-1, GM-CSF, G-CSF, and/or COX-2 production.

In addition to the effects on malignant B cells, immunomodulatory compounds can also upregulate CD40 in normal B cells. Without being limited to a particular theory, this effect may provide a survival signal and protect normal cells during cytotoxic therapies. The protective effect of immunomodulatory compounds on normal cells combined with the increased antitumor effects can potentially increase the Therapeutic Index (T.I.) of the combination therapies. Furthermore, without being limited to a particular theory, CD40 ligation may induce CD95 and can sensitize tumor cells to apoptosis via the c-Abl-dependent activation of p73 and mitigate the resistance of p53-deficient tumor cells to anticancer drug therapy.

In an embodiment, a method of treating cancer in a patient is provided. The method comprises administering to the patient (i) an effective amount of an immunomodulatory compound, and (ii) an effective amount of CD40L, derivative, or fragment thereof. The immunomodulatory compound can be administered, for example, before, concurrently with, or after administration of the CD40L, derivative, or fragment thereof. the immunomodulatory compound can be administered, for example, from about 1, 6, 12, or 24 hours to about 2 days, 4 days, 1 week, or about 2 weeks before administration of the CD40L, derivative, or fragment thereof. the cancer can be, for example, a solid or hematological cancer. the cancer can be, for example, a leukemia, lymphoma or myeloma. The cancer can be leukemia, such as, for example, chronic lymphocytic leukemia (CLL), acute lymphoblastic leukemia (ALL), prolymphocytic leukemia (PLL), hairy cell leukemia, or small lymphocytic leukemia (SLL). The cancer can be lymphoma. the lymphoma can be, for example, Mantle cell lymphoma, splenic lymphoma, hodgkin's lymphoma, mucosal associated lymphoid tissue lymphoma, diffuse small lymphocytic lymphoma, follicular lymphoma, mocytoid B cell lymphoma, Burkitt's lymphoma, AIDS-related lymphoma, diffuse large B-cell lymphoma, lymphomatoid granulomatosis, intravascular lymphomatosis, intravascular lymphoma, cutaneous B-cell lymphoma, or non-hodgkins lymphoma. The cancer can be multiple myeloma. The immunomodulatory compound can be 1-oxo-2-(2,6-dioxopiperidin-3-yl)-4-aminoisoindoline or 1,3-dioxo-2-(2,6-dioxopiperidin-3-yl)-4-aminoisoindoline. The immunomodulatory compound can be administered in an amount between about 2 to about 100 mg/kg. The immunomodulatory compound can be administered orally, parenterally, or topically. The method can further comprise administering an effective amount of an anti-CD40 antibody or a fragment thereof.

In another embodiment, a method of treating a cancer in a patient is provided. The method comprises administering a composition comprising (i) an effective amount of an immunomodulatory compound and (ii) an effective amount of an anti-CD40 antibody or a fragment thereof, where the cancer is leukemia or lymphoma. The immunomodulatory compound can be administered before, concurrently with, or after administration of the anti-CD40 antibody or fragment thereof. the immunomodulatory compound can be administered, for example, from about 1, 6, 12, or 24 hours to about 2 days, 4 days, 1 week, or about 2 weeks before administration of the anti-CD40 antibody or fragment thereof. the cancer can be leukemia, such as, for example, chronic lymphocytic leukemia (CLL), acute lymphoblastic leukemia (ALL), prolymphocytic leukemia (PLL), hairy cell leukemia, or small lymphocytic leukemia (SLL). The cancer can be lymphoma, such as, for example, Mantle cell lymphoma, splenic lymphoma, hodgkin's lymphoma, mucosal associated lymphoid tissue lymphoma, diffuse small lymphocytic lymphoma, follicular lymphoma, mocytoid B cell lymphoma, Burkitt's lymphoma, AIDS-related lymphoma, diffuse large B-cell lymphoma, lymphomatoid granulomatosis, intravascular lymphomatosis, intravascular lymphoma, cutaneous B-cell lymphoma, or non-hodgkins lymphoma. The immunomodulatory compound can be, for example, 1-oxo-2-(2,6-dioxopiperidin-3-yl)-4-aminoisoindoline or 1,3-dioxo-2-(2,6-dioxopiperidin-3-yl)-4-aminoisoindoline. The immunomodulatory compound can be administered, for example, in an amount of between about 2 to about 100 mg/kg. The immunomodulatory compound can be administered, for example, orally, parenterally, or topically.

In some embodiments, the immunomodulatory compound can be, for example, a compound of formula I, II, III, IV, V, VI, VII, VIII, IX, X, XI, XII, XIII, XIV, XV, XVI, XVII, XVIII or a pharmaceutically acceptable salt, solvate, or stereoisomer thereof:

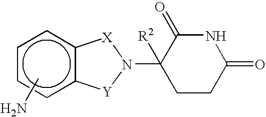

wherein:
one of X and Y is C═O, the other of X and Y is C═O or CH₂;
R² is hydrogen or lower alkyl;

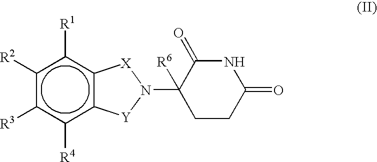

wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms, or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino;

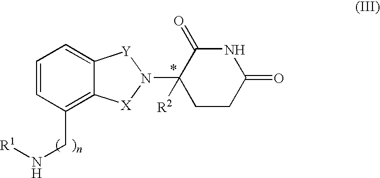

wherein:
one of X and Y is C═O and the other is CH₁₂ or C═O;
R¹ is H, (C₁-C₈)alkyl, (C₃₋₇)cycloalkyl, (C₂-C₈)alkenyl, (C₂-C₈)alkynyl, benzyl, aryl, (C₀-C₄)alkyl C₁-C₆)heterocycloalkyl, (C₀-C₄)alkyl C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, F, benzyl, (C₁-C₉)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈) alkynyl, benzyl, aryl, (C₀-C₄)alkyl C₁-C₆)heterocycloalkyl, (C₀-C₄)alkyl-(C₂-C₅)heteroaryl, (C₀-C₉)alkyl-N(R⁶)₂, (C₁-C₈)alkyl-OR⁵, (C₁-C₈)alkyl-C(O)OR⁵, (C₁-C₈)alkyl-O(CO)R⁵ or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl C₁-C₆)heterocycloalkyl, or (C₀-C₄)alkyl C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* is a chiral-carbon center;

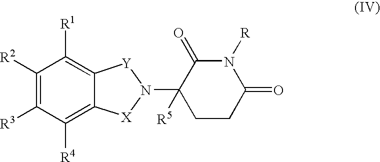

wherein:
one of X and Y is C═O and the other is CH₂ or C═O;
R is H or CH₂OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CⁿH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;
R¹⁰ is hydrogen, alkyl of to 8 carbon atoms, or phenyl; and
* represents a chiral-carbon center;

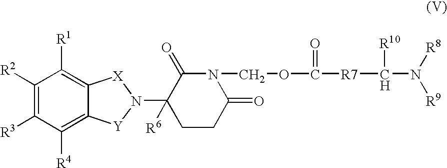

wherein:
one of X and Y is C═O and the other of X and Y is C═O or CH₂;
(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzo, chloro, or fluoro;
R⁷ is m-phenylene or p-phenylene or —(CⁿH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and

R¹⁰ is hydrogen, alkyl of to 8 carbon atoms, or phenyl;

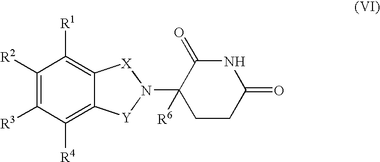

wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and
R⁶ is hydrogen, alkyl of 1 to 8 carbon atoms, benzo, chloro, or fluoro;

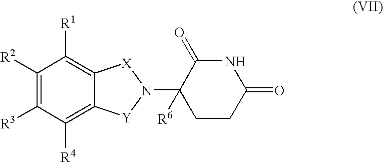

wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and
R⁶ is alkyl of 1 to 8 carbon atoms, benzo, chloro, or fluoro;

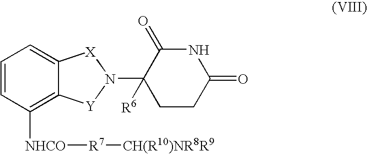

wherein:
one of X and Y is C═O and the other of X and Y is C═O or CH₂;
R⁶ is hydrogen, alkyl of 1 to 8 carbon atoms, benzyl, chloro, or fluoro;
R⁷ is m-phenylene, p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and
R¹⁰ is hydrogen, alkyl of 1 to 8 carbon atoms, or phenyl;

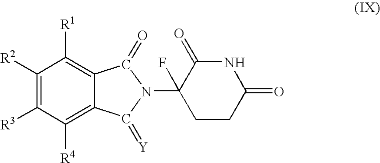

wherein:
Y is oxygen or H² and
each of R¹, R², R³, and R⁴, independently of the others, is hydrogen, halo, alkyl of 1 to 4 carbon atoms, alkoxy of 1 to 4 carbon atoms, or amino;

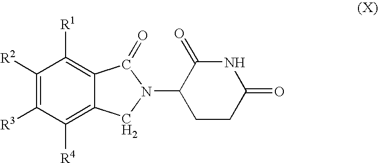

wherein:
each of R¹, R², R³, and R⁴, independently of the others, is halo, alkyl of 1 to 4 carbon atoms, or alkoxy of 1 to 4 carbon atoms;

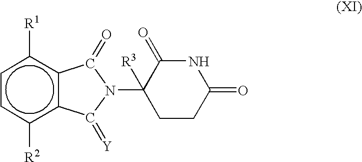

wherein:
Y is oxygen or H₂,
a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and
R³ is hydrogen, alkyl, or benzyl;

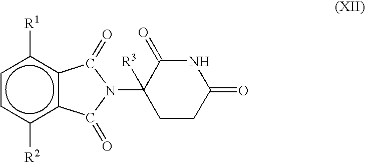

wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl;

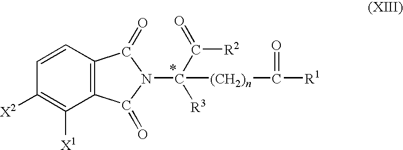

wherein:
when n is not zero and R¹ is not the same as R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is hydrogen, alkyl of one to six carbons, halo, or haloalkyl;
Z is hydrogen, aryl, alkyl of one to six carbons, formyl, or acyl of one to six carbons; and
n has a value of 0, 1, or 2;
   provided that if X¹ is amino, and n is 1 or 2, then R¹ and R² are not both hydroxy;

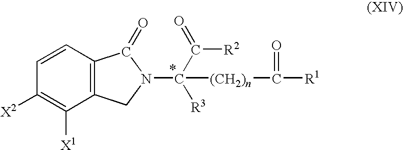

wherein:
when n is not zero and R¹ is not R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is alkyl of one to six carbons, halo, or hydrogen;
Z is hydrogen, aryl or an alkyl or acyl of one to six carbons; and
n has a value of 0, 1, or 2;

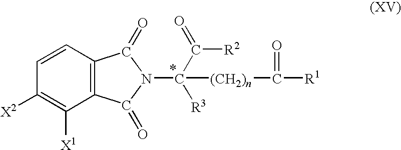

wherein:
when n is not zero and R¹ is not R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is alkyl of one to six carbons, halo, or hydrogen;
Z is hydrogen, aryl, or an alkyl or acyl of one to six carbons; and
n has a value of 0, 1, or 2;

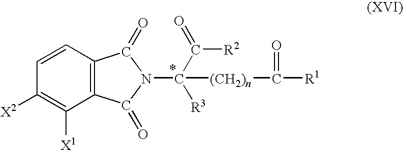

wherein:
one of X¹ and X² is nitro, or NH-Z, and the other of X¹ or X² is hydrogen;
each of R¹ and R², independent of the other, is hydroxy or NH
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality;

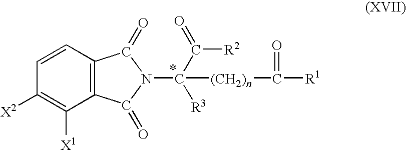

wherein:
one of X¹ and X² is alkyl of one to six carbons;
each of R¹ and R², independent of the other, is hydroxy or NH-Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality;

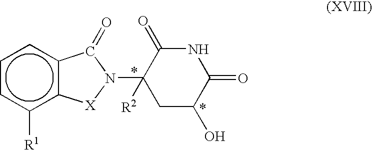

wherein:
the * carbons are centers of chirality;
X is —C(O)— or —CH₂—;
R¹ is alkyl of 1 to 8 carbon atoms or —NHR³;
R² is hydrogen, alkyl of 1 to 8 carbon atoms, or halogen; and
R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴, wherein
R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

In an embodiment, the immunomodulatory compound can be, for example, 1-oxo-2-(2,6-dioxopiperidin-3-yl)-4-aminoisoindoline or 1,3-dioxo-2-(2,6-dioxopiperidin-3-yl)-4-aminoisoindoline.

### 5.1 Definitions

As used herein, and unless otherwise specified, the terms “treat,” “treating” and “treatment” refer to an action that occurs while a patient is suffering from the specified cancer, which reduces the severity of the cancer, or retards or slows the progression of the cancer.

As used herein, unless otherwise specified, the terms “prevent,” “preventing” and “prevention” refer to an action that occurs before a patient begins to suffer from the specified cancer, which inhibits or reduces the severity of the cancer.

As used herein, and unless otherwise indicated, the terms “manage,” “managing” and “management” encompass preventing the recurrence of the specified cancer in a patient who has already suffered from the cancer, and/or lengthening the time that a patient who has suffered from the cancer remains in remission. The terms encompass modulating the threshold, development and/or duration of the cancer, or changing the way that a patient responds to the cancer.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

As used herein, and unless otherwise specified, the term “prophylactically effective amount” of a composition is an amount sufficient to prevent cancer, or one or more symptoms associated with cancer, or prevent its recurrence. The term “prophylactically effective amount” can encompass an amount that improves overall prophylaxis or enhances the prophylactic efficacy of another prophylactic agent.

An improvement in the cancer or cancer-related disease can be characterized as a complete or partial response. “complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The terms “treatment” and “prevention” contemplate both a complete and a partial response.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth. Benign tumors generally remain localized. Malignant tumors are collectively termed cancers. The term “malignant” generally means that the tumor can invade and destroy neighboring body structures and spread to distant sites to cause death.

The terms “cancer” and “cancerous” refer to or describe the physiological condition in mammals that is typically characterized by unregulated cell growth. Examples of cancer include, but are not limited to, lymphoma and leukemia, and solid tumors.

By “B cell-related cancer” or “cancer of B-cell lineage” is intended any type of cancer in which the dysregulated or unregulated cell growth is associated with B cells. In one embodiment, the B cell related cancer is a hematological or solid cancer.

The term “antibody” is used herein in the broadest sense and covers fully assembled antibodies, antibody fragments which retain the ability to specifically bind to the antigen e.g. Fab, F(ab′)₂, Fv, and other fragments, single chain antibodies, diabodies, antibody chimeras, hybrid antibodies, bispecific antibodies, humanized antibodies, and the like), and recombinant peptides comprising the forgoing. The term “antibody” covers both polyclonal and monoclonal antibodies.

The term “monoclonal antibody” (and “mAb”) as used herein refers to an antibody obtained from a substantially homogeneous population of antibodies, i.e., the individual antibodies comprising the population are identical except for possible naturally occurring mutations that can be present in minor amounts. The term is not limited regarding the species of the antibody and does not require production of the antibody by any particular method.

As noted above, the term “antibody” as used herein also encompasses chimeric antibodies. “Chimeric” antibodies encompass antibodies that are derived using recombinant deoxyribonucleic acid techniques and which may comprise both human (including immunologically “related” species, e.g., chimpanzee) and non-human components. Thus, the constant region of the chimeric antibody may be substantially identical to the constant region of a natural human antibody; the variable region of the chimeric antibody can be derived from a non-human source and has the desired antigenic specificity to the antigen of interest.

The term “antibody” as used herein encompasses humanized antibodies. “Humanized” antibodies encompass forms of antibodies that may contain minimal sequence derived from non-human immunoglobulin sequences. Typically, humanized antibodies are human immunoglobulins (recipient antibody) in which residues from a hypervariable region (also known as complementarity determining region or CDR) of the recipient are replaced by residues from a hypervariable region of a non-human species (donor antibody) such as, but not limited to, mouse, rat, rabbit, or nonhuman primate having the desired specificity, affinity, and capacity.

The term “antibody” as used herein also encompasses antibody fragments that can bind antigen. “Antibody fragments” comprise a portion of an intact antibody, for example, the antigen-binding or variable region of the intact antibody. Examples of antibody fragments include, but are not limited to, Fab, F(ab′)₂, and Fv fragments; diabodies; linear antibodies (Zapata et al. (1995) Protein Eng. 10: 1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

Without being limited by a particular theory, it was found that CD40 can be upregulated by immunomodulatory compounds in malignant B cells, causing them to be more sensitive to CD40 ligation therapy. In some aspects, it was discovered that pre-treatment with immunomodulatory compounds can cause tumor cells to be more susceptible to the apoptotic effects of CD40 ligation. Immunomodulatory compounds also upregulate CD40L in T cells, which could potentially increase the killing of tumor cells by the patient's own lymphocytes.

### 5.2 Compounds

5.2.1 Immunomodulatory Compounds

Any suitable immunomodulatory compounds can be used in the combination therapy methods described herein. Exemplary immunomodulatory compounds that can be administered include but are not limited to N-{[2-(2,6-dioxo(3-piperidyl)-1,3-dioxoisoindolin-4-yl]methyl}cyclopropyl-carboxamide; 3-[2-(2,6-dioxo-piperidin-3-yl)-1,3-dioxo-2,3-dihydro-1H-isoindol-4-ylmethyl]-1,1-dimethyl-urea; (−)-3-(3,4-Dimethoxy-phenyl)-3-(1-oxo-1,3-dihydro-isoindol-2-yl)-propionamide; (+)-3-(3,4-Dimethoxy-phenyl)-3-(1-oxo-1,3-dihydro-isoindol-2-yl)-propionamide; (−)-{2-[1-(3-ethoxy-4-methoxyphenyl)-2-methylsulfonylethyl]-4-acetylaminoisoindoline-1,3-dione}; (+)-{2-[1-(3-ethoxy-4-methoxyphenyl)-2-methylsulfonylethyl]-4-acetylaminoisoindoline-1,3-dione}; Difluoro-methoxy SelCIDs; 1-phthalimido-1-(3,4-diethoxyphenyl)ethane; 3-(3,4-dimethoxyphenyl)-3-(3,5-dimethoxyphenyl)acrylo nitrile; 1-oxo-2-(2,6-dioxopiperidin-3-yl)-4-aminoisoindoline; 1,3-dioxo-2-(2,6-dioxopiperidin-3-yl)-4-aminoisoindoline; 4-amino-2-(3-methyl-2,6-dioxo-piperidine-3-yl)-isoindole-1,3-dione; 3-(3-acetoamidophthalimido)-3-(3-ethoxy-4-methoxyphenyl)-N-hydroxypropionamide; 1-oxo-2-(2,6-dioxopiperidin-3-yl)-4-methylisoindoline; Cyclopropyl-N-{2-[(1S)-1-(3-ethoxy-4-methoxyphenyl)-2-(methylsulfonyl)ethyl]-3-oxoisoindoline-4-yl}carboxamide; Substituted 2-(3-hydroxy-2,6-dioxopiperidin-5-yl) isoindoline; N-[2-(2,6-Dioxo-piperidin-3-yl)-1,3-dioxo-2,3-dihydro-1H-isoindol-5-ylmethyl]-4-trifluoromethoxybenzamide; (S)-4-chloro-N-((2-(3-methyl-2,6-dioxopiperidin-3-yl)-1,3-dioxoisoindolin-5-yl)methyl)benzamide; Pyridine-2-carboxylic acid [2-[(3S)-3-methyl-2,6-dioxo-piperidin-3-yl]-1,3-dioxo-2,3-dihydro-1H-isoindol-5-ylmethyl]-amide; (S)-N-((2-(3-methyl-2,6-dioxopiperidin-3-yl)-1,3-dioxoisoindolin-5-yl)methyl)-4-(trifluoromethyl)benzamide; 3-(2,5-dimethyl-4-oxo-4H-quinazolin-3-yl)-piperidine-2,6-dione, and the like.

Without being limited by theory, immunomodulatory compounds disclosed herein may be potent co-stimulators of T cells and increase cell proliferation dramatically in a dose dependent manner. Immunomodulatory compounds disclosed herein may also have a greater co-stimulatory effect on the CD8⁺ T cell subset than on the CD4⁺ T cell subset. In addition, the compounds may have anti-inflammatory properties against myeloid cell responses, yet efficiently co-stimulate T cells to produce greater amounts of IL-2, IFN-γ, and to enhance T cell proliferation and CD8⁺ T cell cytotoxic activity. Further, without being limited by a particular theory, immunomodulatory compounds disclosed herein may be capable of acting both indirectly through cytokine activation and directly on Natural Killer (“NK”) cells and Natural Killer T (“NKT”) cells, and increase the ability of NK cells to produce beneficial cytokines such as, but not limited to, IFN-γ, and to enhance NK and NKT cell cytotoxic activity.

Specific examples of immunomodulatory compounds include cyano and carboxy derivatives of substituted styrenes such as those disclosed in U.S. Pat. No. 5,929,117; 1-oxo-2-(2,6-dioxo-3-fluoropiperidin-3-yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), substituted 2-(2,6-dioxopiperidin-3-yl) phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. patent publication no. 2003/0045552, U.S. Pat. No. 7,091,353, and International Application No. PCT/US01/50401 (International Publication No. WO 02/059106). US patent publication no. 2006/0205787 describes 4-amino-2-(3-methyl-2,6-dioxopiperidin-3-yl)-isoindole-1,3-dione compositions. US patent publication no. 2007/0049618 describes isoindole-imide compounds. The entireties of each of the patents and patent applications identified herein are incorporated by reference. In one embodiment, immunomodulatory compounds do not include thalidomide.

Various immunomodulatory compounds disclosed herein contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of a particular immunomodulatory compounds may be used. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, 1N, 1972), each of which is incorporated by reference herein in its entirety.

Immunomodulatory compounds provided herein include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference.

These compounds have the structure:

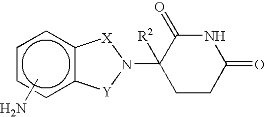

### I

in which one of X and Y is C═O, the other of X and Y is C═O or CH₂, and R² is hydrogen or lower alkyl, in particular methyl. Specific immunomodulatory compounds include, but are not limited to:

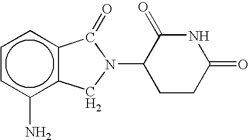

1-oxo-2-(2,6-dioxopiperidin-3-yl)-4-aminoisoindoline;

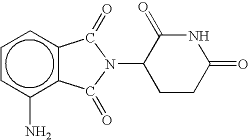

1,3-dioxo-2-(2,6-dioxopiperidin-3-yl)-4-aminoisoindoline;

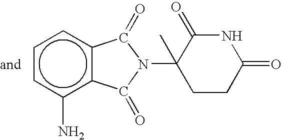

1,3-dioxo-2-(3-methyl-2,6-dioxopiperidin-3-yl)-4-aminoisoindole, and optically pure isomers thereof.

The compounds can be obtained via standard, synthetic methods (see e.g., U.S. Pat. No. 5,635,517, incorporated herein by reference). The compounds are also available from Celgene Corporation, Warren, N.J.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula:

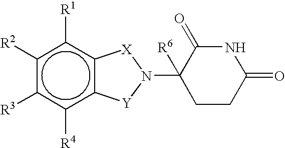

in which:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;
provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas:

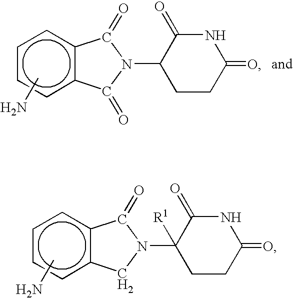

wherein R¹ is hydrogen or methyl. In a separate embodiment, the invention encompasses the use of enantiomerically pure forms (e.g. optically pure (R) or (S) enantiomers) of these compounds.

Still other specific immunomodulatory compounds disclosed herein belong to a class of isoindole-imides disclosed in U.S. Pat. No. 7,091,353, U.S. Patent Publication No. 2003/0045552, and International Application No. PCT/US01/50401 (International Publication No. WO 02/059106), each of which are incorporated herein by reference. Representative compounds are of formula:

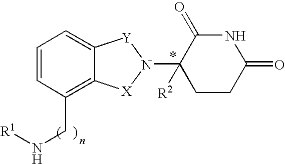

and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl C₁-C₆)heterocycloalkyl, (C₀-C₄)alkyl C₂-C₅)heteroaryl, C(O)R³, C(S)R³, C(O)OR⁴, (C₁-C₈)alkyl-N(R⁶)2, (C₁-C₈)alkyl-OR⁵, (C₁-C₈)alkyl-C(O)OR⁵, C(O)NHR³, C(S)NHR³, C(O)NR³R³, C(S)NR³R³, or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl C₁-C₆)heterocycloalkyl, (C₀-C₄)alkyl C₂-C₅)heteroaryl, (C₀-C₈)alkyl-N(R⁶)₂, (C₁-C₉)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl; each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* represents a chiral-carbon center.

In specific compounds of the formula, when n is 0 then R¹ is (C₃-C₇)cycloalkyl, (C₂-C₈)alkenyl, (C₂-C₈)alkynyl, benzyl, aryl, (C₀-C₄)alkyl)-C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and
R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

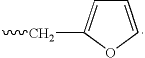

In another embodiment of the compounds of formula II, R¹ is

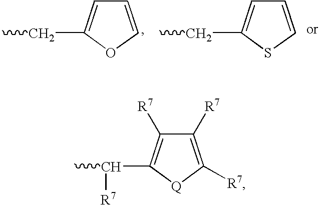

wherein Q is O or S, and each occurrence of R⁷ is independently H,(C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl C₂-C₅)heteroaryl, (C₀-C₈)alkyl-N(R⁶⁰)2, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific immunomodulatory compounds disclosed herein belong to a class of isoindole-imides disclosed in U.S. Pat. No. 6,555,554, International Publication No. WO 98/54170, and U.S. Pat. No. 6,395,754, each of which is incorporated herein by reference. Representative compounds are of formula:

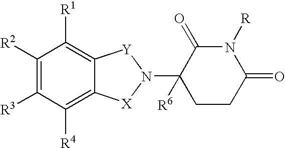

and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;

### R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;

### R′ is R⁷—CHR¹⁰—N(R⁸R⁹);

R⁷ is m-phenylene or p-phenylene or —(CnH2n)- in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—;
R¹⁰ is hydrogen, alkyl of to 8 carbon atoms, or phenyl; and
* represents a chiral-carbon center.

Other representative compounds are of formula:

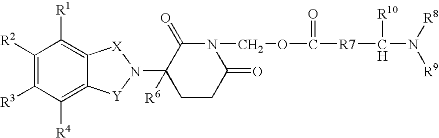

wherein:
one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CnH2n)- in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and

R¹⁰ is hydrogen, alkyl of to 8 carbon atoms, or phenyl.

Other representative compounds are of formula:

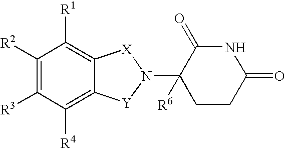

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R²R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

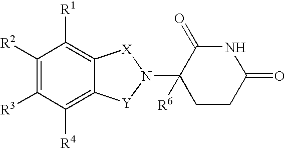

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

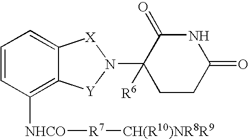

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CnH2n)- in which n has a value of 0 to 4; each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Other specific immunomodulatory compounds are 1-oxo-2-(2,6-dioxo-3-fluoropiperidin-3-yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

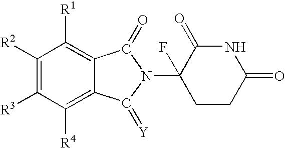

wherein:
Y is oxygen or H₂ and
each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds are the tetra substituted 2-(2,6-dioxopiperidin-3-yl)-1-oxoisoindolines described in U.S. Pat. No. 5,798,368, which is incorporated herein by reference. Representative compounds are of formula:

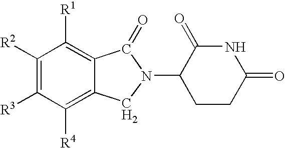

wherein each of R¹, R², R³, and R⁴, independently of the others, is halo, alkyl of 1 to 4 carbon atoms, or alkoxy of 1 to 4 carbon atoms.

Other specific immunomodulatory compounds are 1-oxo and 1,3-dioxo-2-(2,6-dioxopiperidin-3-yl) isoindolines disclosed in U.S. Pat. No. 6,403,613, which is incorporated herein by reference. Representative compounds are of formula:

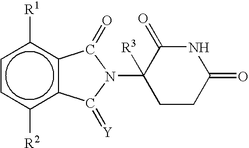

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

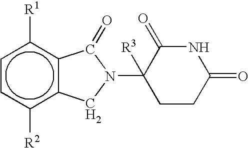

wherein
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

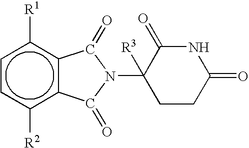

wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl.

Other specific immunomodulatory compounds disclosed herein are 1-oxo and 1,3-dioxoisoindolines substituted in the 4- or 5-position of the indoline ring described in U.S. Pat. No. 6,380,239 and U.S. Pat. No. 7,244,759, both of which are incorporated herein by reference. Representative compounds are of formula:

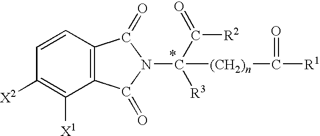

in which the carbon atom designated C* constitutes a center of chirality (when n is not zero and R¹ is not the same as R²); one of X¹ and X² is amino, nitro, alkyl of one to six carbons, or NH-Z, and the other of X¹ or X² is hydrogen; each of R¹ and R² independent of the other, is hydroxy or NH-Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

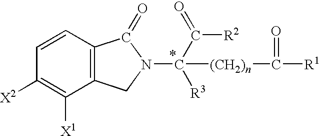

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

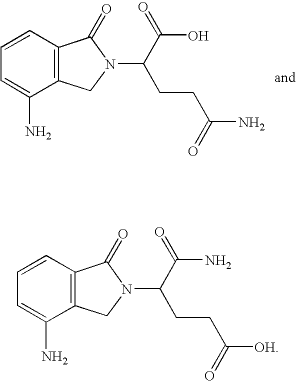

Other representative compounds are of formula:

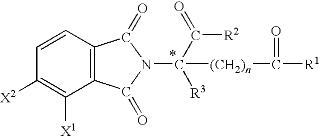

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stercoisomers thereof:

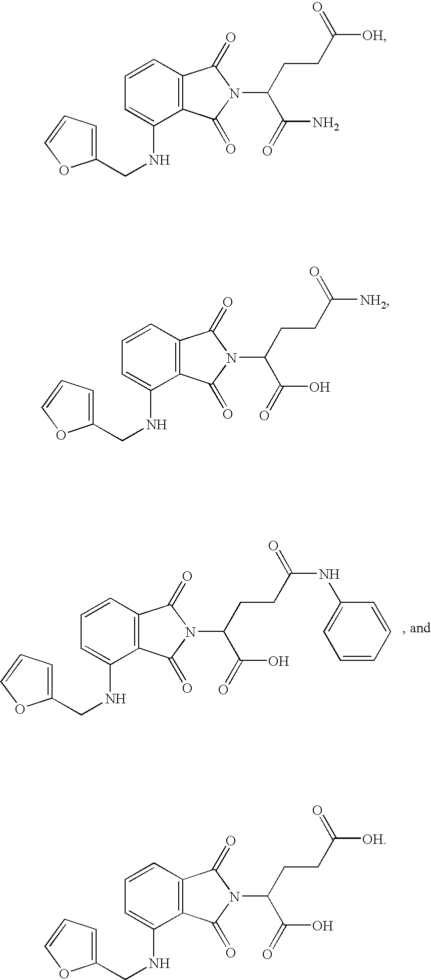

Other specific examples of the compounds are of formula:

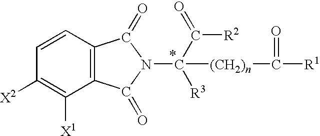

wherein:

one of X¹ and X² is nitro, or NH-Z, and the other of X¹ or X² is hydrogen;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Other representative compounds are of formula:

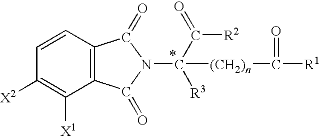

wherein:

one of X¹ and X² is alkyl of one to six carbons;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds are isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

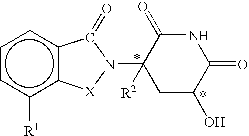

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

All of the compounds described can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques. Additional information on immunomodulatory compounds, their preparation, and use can be found, for example, in U.S. Patent Application Publication Nos. US20060188475, US20060205787, and US20070049618, each of which is incorporated by reference herein in its entirety.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

5.2.2 Anti-CD40 Antibodies

In some embodiments, an immunomodulatory compound can be administered in combination with an anti-CD40 antibody to effectively treat cancer, such as a B-cell cancer. In some embodiments, the anti-CD40 antibody is a full length antibody. In additional embodiments, the antibody is a partial length antibody or fragment of an antibody.

In some embodiments, an immunomodulatory compound can be administered in combination with an anti-CD40 antibody or fragment thereof to target a malignant B cell for destruction in an individual having a B-cell related cancer.

Many types of antibodies to CD40 can be used. In some embodiments, the anti-CD40 antibody can be a monoclonal antibody. A monoclonal antibody (mAb), as used herein, generally refers to an antibody obtained from a substantially homogeneous population of antibodies, i.e., the individual antibodies comprising the population are identical except for possible naturally occurring mutations that can be present in minor amounts. In other embodiments, the anti-CD40 antibody can be a polyclonal antibody.

In contrast to polyclonal antibody preparations, which typically include different antibodies directed against different antigenic determinants (epitopes), each monoclonal antibody is generally directed against a single determinant (epitope) on the antigen. An epitope is generally the portion of an antigenic molecule to which an antibody is produced and to which the antibody can bind. Epitopes can comprise linear amino acid residues (i.e., residues within the epitope are arranged sequentially one after another in a linear fashion), non-linear amino acid residues (referred to herein as “non-linear epitopes”; these epitopes are not arranged sequentially), or both linear and non-linear amino acid residues. A anti-CD40 monoclonal antibody suitable for use in the methods provided herein can be capable of specifically binding to an epitope on CD40 expressed on the surface of a cell.

When the composition comprises an anti-CD40 monoclonal antibody, any suitable method for obtaining monoclonal antibodies can be used. Anti-CD40 monoclonal antibodies can be made by the hybridoma method first described by Kohler et al. (1975) Nature 256:495, incorporated herein by reference, or can be made by recombinant DNA methods. Monoclonal antibodies can also be isolated from antibody phage libraries generated using the techniques described in, for example, McCafferty et al. (1990) Nature 348:552-554 (1990), which is incorporated by reference herein in its entirety. Further, Clackson et al. (1991) Nature 352:624-628 and Marks et al. (1991) J Mol. Biol. 222:581-597, each of which is incorporated by reference herein in its entirety, describe the isolation of murine and human antibodies, respectively, using phage libraries. Methods of producing high affinity (nM range) human antibodies by chain shuffling (Marks et al. (1992) Bio/Technology 10:779-783, which is incorporated by reference herein in its entirety), as well as combinatorial infection and in vivo recombination as a strategy for constructing very large phage libraries (Waterhouse et al. (1993) Nucleic. Acids Res. 21:2265-2266, which is incorporated by reference herein in its entirety) can be used. Alternatively, the anti-CD40 monoclonal antibody can be obtained commercially.

5.2.3 CD40 Ligand

The native CD40 ligand (CD40L; CD 154) is a transmembrane protein which is expressed on certain types of helper T cells. While not being limited to a particular theory, CD40L is recognized by CD40 on the B cell surface. The interaction between CD40 ligand and CD40 results in the activation of B cells by helper T cells. The activated B cells can then proliferate and differentiate into memory or antibody-secreting effector cells.

Additionally, while not being limited to a particular theory, the CD40 ligand is often involved in responses to infection. TH1 effector cells can display the CD40 ligand, which binds to CD40 on a macrophage. This signal, along with IFN-γ, which binds to IFN-γ receptors on the macrophage surface, results in macrophage activation.

The combination of immunomodulatory compounds with CD40L therapy and/or antiCD40 therapy may make cells more sensitive to therapy. Without being limited to a particular theory, by upregulating the expression of CD40 on tumor cells, immunomodulatory compounds can make these cells more susceptible to anti-CD40 therapy and/or cytotoxic T cells while at the same time maintaining and improving the viability of normal B cells.

In some embodiments, a CD40L protein, polypeptide or fragment thereof can be administered to a patient in combination with administration of an immunomodulatory compound. In yet further embodiments, an immunomodulatory compound, CD40L, and a CD40 antibody are administered. The CD40L or fragment thereof can be isolated from mammalian sources, such as human blood, or can be prepared, for example, using recombinant technologies, followed by production in non-mammalian systems such as yeast or bacteria. In some embodiments, purified CD40L is obtained commercially. In some embodiments, a truncated form or soluble form of CD40L is used. In some embodiments, a soluble form of CD40L can be more easily administered to the patient than the native, full-length form that has hydrophobic membrane spanning regions. In other embodiments, the CD40L can be administered by expression on the surface of a cell, then administering the cells to the patient. Alternatively, CD40L can be delivered endogenously, for example, by gene therapy methods that are capable of upregulating CD40L on the cell surface.

The anti-CD40 antibody and CD40L that can be added in combination with the immunomodulatory compound can also encompass mutants, derivatives (e.g., modified forms), or truncated forms of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins.

In some embodiments, the compositions comprise variants of anti-CD40 antibody or CD40L. In some embodiments, variants have amino acid sequences that have at least 70% or 75% sequence identity, at least 80% or 85% sequence identity, or at least 90%, 95%, or 98% sequence identity to at least a portion of the amino acid sequence of anti-CD40 antibody or CD40L. A variant can, for example, differ from anti-CD40 antibody or CD40L, by as few as 1 to 15 amino acid residues, as few as 1 to 10 amino acid residues, such as 6-10, as few as 5, as few as 4, 3, 2, or even 1 amino acid residue.

### 5.3 Methods of Treatment

5.3.1 Cancer

Many types of cancer can be treated using the combinations of immunomodulatory compounds plus other compounds as disclosed herein. Specific examples of cancer include, but are not limited to: cancers of the skin, such as melanoma; lymph node; breast; cervix; uterus; gastrointestinal tract; lung; ovary; prostate; colon; rectum; mouth; brain; head and neck; throat; testes; kidney; pancreas; bone; spleen; liver; bladder; larynx; nasal passages; and AIDS-related cancers. Methods provided herein can also be used to follow the treatment of cancers of the blood and bone marrow, such as multiple myeloma and acute and chronic leukemias, for example, lymphoblastic, myelogenous, lymphocytic, myelocytic leukemias, and myelodysplastic syndromes including but not limited to 5 q minus syndrome, or myelodysplastic syndromes associated with other cytogenic abnormalities, and the like. The methods provided herein can be used for managing either primary or metastatic tumors.

In some embodiments, the cancer to be treated is multiple myeloma. In other embodiments, the cancer to be treated is not multiple myeloma.

Other specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma (localized melanoma, including, but not limited to, ocular melanoma), malignant mesothelioma, malignant pleural effusion mesothelioma syndrome, peritoneal carcinoma, papillary serous carcinoma, gynecologic sarcoma, soft tissue sarcoma, ovarian cancer, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, leiomyoma, and the like. In a specific embodiment, the cancer is metastatic. In another embodiment, the cancer is refractory or resistant to chemotherapy or radiation.

In some embodiments, the cancer is a solid or hematological cancer. Examples include, but are not limited to: ovarian cancer, prostate cancer, pancreatic cancer, leukemias including, but not limited to, chronic lymphocytic leukemia, chronic myelocytic leukemia, acute lymphoblastic leukemia (ALL), acute myelogenous leukemia (AML) and acute myeloblastic leukemia; lymphomas including, but are not limited to, Hodgkin's and non-Hodgkin's lymphomas, including all of the subtypes thereof; and myelomas including, but not limited to, multiple myeloma. In some embodiments, the hematological cancer is multiple myeloma.

Exemplary types of leukemia include, but are not limited to chronic lymphocytic leukemia (CLL), acute lymphoblastic leukemia (ALL), prolymphocytic leukemia (PLL), hairy cell leukemia, and small lymphocytic leukemia (SLL).

Exemplary types of lymphoma include but are not limited to Mantle cell lymphoma, splenic lymphoma, hodgkin's lymphoma, mucosal associated lymphoid tissue lymphoma, diffuse small lymphocytic lymphoma, follicular lymphoma, mocytoid B cell lymphoma, Burkitt's lymphoma, AIDS-related lymphoma, diffuse large B-cell lymphoma, lymphomatoid granulomatosis, intravascular lymphomatosis, intravascular lymphoma, cutaneous B-cell lymphoma, and non-hodgkins lymphoma.

5.3.2 Methods of Administration

Methods provided herein comprise administering one or more immunomodulatory compounds, or a pharmaceutically acceptable salt, solvate, stereoisomer, or prodrug thereof, in combination with an anti-CD40 antibody and/or CD40 ligand to a patient (e.g., a human) suffering, or likely to suffer, from a cancer-related disease or disorder.

Any of the components of the composition can be administered together or separately. Each of the components can be administered by any suitable means. In some embodiments, at least a part of the formulation is administered using intravenous administration. In some embodiments, intravenous administration can occur by infusion over a period of about less than 1 hour to about 10 hours (less than 1, 2, 3, 4, 5, 6, 7, 8, 9, or 10 hours). Subsequent infusions can be administered over a period of about less than 1 to about 6 hours or more, including, for example, about 1 to about 4 hours, about 1 to about 3 hours, or about 1 to about 2 hours or less than an hour. Alternatively, a dose can be administered subcutaneously or by other means.

In some embodiments, at least a part of the formulation is administered orally. Pharmaceutical compositions that are suitable for oral administration can be presented as in several types of forms, such as, but not limited to, tablets, caplets, capsules, and liquids. In some embodiments, oral administration of a component of the composition can occur prior to, after, or during the administration of the other components. For example, oral administration of at least one of the components can occur 2 weeks, 1 week, 3 days, one day, 12 hours, 1 hour, or 30 minutes prior to or after administration of the other components of the composition. Oral administration of at least one component of the composition can occur several times per day, daily, once, every other day, once weekly, and the like.

A single dose of the immunomodulatory compound to be administered can be, for example, in the range from about 0.1 mg/kg of patient body weight to about 300 mg/kg, from about 0.1 mg/kg to about 200 mg/kg, from about 0.1 mg/kg to about 50 mg/kg, from about 0.5 mg/kg to about 200 mg/kg, from about 1 mg/kg to about 150 mg/kg, from about 3 mg/kg to about 300 mg/kg, from about 3 mg/kg to about 200 mg/kg, from about 5 mg/kg to about 100 mg/kg, from about 10 mg/kg to about 50 mg/kg.

Thus, for example, the immunomodulatory compound dose can be 0.1 mg/kg, 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 50 mg/kg, 75 mg/kg, 100 mg/kg, 150 mg/kg, 200 mg/kg, 250 mg/kg, 300 mg/kg, or other such doses falling within the range of about 0.1 mg/kg to about 300 mg/kg.

Similarly, a single dose of the anti-CD40 antibody to be administered can be, for example, in the range from about 0.1 mg/kg to about 100 mg/kg, from about 0.1 mg/kg to about 75 mg/kg, from about 0.1 mg/kg to about 50 mg/kg, from about 0.5 mg/kg to about 50 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg.

Thus, for example, the anti-CD40 antibody dose can be 0.1 mg/kg, 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 40 mg/kg, 50 mg/kg, 75 mg/kg, 100 mg/kg, or other such doses falling within the range of about 0.1 mg/kg to about 100 mg/kg.

Similarly, a single dose of CD40 ligand to be administered can be, for example, in the range from about 0.1 mg/kg to about 100 mg/kg, from about 0.1 mg/kg to about 75 mg/kg, from about 0.1 mg/kg to about 50 mg/kg, from about 0.5 mg/kg to about 50 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg.

Thus, for example, the CD40 ligand dose can be 0.1 mg/kg, 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 40 mg/kg, 50 mg/kg, 75 mg/kg, 100 mg/kg, or other such doses falling within the range of about 0.1 mg/kg to about 100 mg/kg.

Administration of the immunomodulatory compound “in combination with” one or more further therapeutic agents includes simultaneous (concurrent) and consecutive administration in any order. Thus, the immunomodulatory compound can be administered at the same time, or prior to, or after one or more of the compounds in the combination. For example, the composition to be administered can contain all of the ingredients in the combination. Alternatively, one or more of the compounds in the composition can be administered before or after the other compounds. In one embodiment, the immunomodulatory compound can be administered at the same time as the other components in the composition. In another embodiment, the immunomodulatory compound can be administered from about 0, 10, 30, to about 60 minutes or more after administration of at least one of the other compounds in the composition. In other embodiments, the immunomodulatory compound can be administered from about 1, 6, 12, or 24 hours to about 2 days, 4 days, 1 week, or about 2 weeks after administration of at least one of the other compounds in the composition.

In some embodiments, it is advantageous to pretreat the patient with the immunomodulatory compound prior to administration of a second compound, particularly so that cellular changes (such as protein expression) effected by the immunomodulatory compound can already be present when the second compound is administered. In an embodiment, the immunomodulatory compound can be administered from about 0, 10, 30, to about 60 minutes before administration of an anti-CD40 antibody and/or CD40L. In another embodiment, the immunomodulatory compound can be administered from about 1, 6, 12, or 24 hours to about 2 days, 4 days, 1 week, or about 2 weeks before administration of an anti-CD40 antibody and/or CD40L.

The immunomodulatory compound can be administered by the same route, or by a different route, than the other compound or compounds in the combination. For example, some of the components of the composition can be administered orally, while others are administered intravenously. In additional embodiments, some of the components are administered by subcutaneous injection, while other components are administered by infusion.

### 5.4 Pharmaceutical Compositions and Dosage Forms

The composition of an immunomodulatory compound in combination with an anti-CD40 antibody and/or CD40L can be formulated into desired dosage forms. For example, single or multiple unit dosage forms can be prepared.

The compositions can be formulated to be suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a cancer-related disease or disorder may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same cancer-related disease or disorder. These and other ways in which specific dosage forms encompassed by this invention will vary from one another will be readily apparent to those skilled in the art. (See, e.g., Remington's Pharmaceutical Sciences, 18th ed., Mack Publishing, Easton Pa. (1990)).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients, or when exposed to water.

Further provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. (>See, e.g., Jens T. Carstensen, Drug Stability. Principles & Practice, 2d. Ed., Marcel Dekker, NY, N.Y., 1995, pp. 379-80, which is incorporated by reference herein in its entirety). In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, in some embodiments, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, strip packs, and the like.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, salt buffers, and the like.

The amounts and specific types of active ingredients in a dosage form may differ depending on factors such as, but not limited to, the route by which it is to be administered to patients. Thus, in some embodiments, typical dosage forms comprise an immunomodulatory compound or a pharmaceutically acceptable salt, solvate, stereoisomer, or prodrug thereof in an amount of from about 1 mg to about 250 mg. In some embodiments, dosage forms can comprise an immunomodulatory compound or a pharmaceutically acceptable salt, solvate, stereoisomer, or prodrug thereof in an amount of about 2, 5, 7.5, 10, 12.5, 15, 17.5, 20, 25, 50, 100, 150 or 200 mg. Typical dosage forms comprise the second active ingredient, such as anti-CD40 antibody or CD40L, in an amount of from about 0.1 to about 1000 mg, from about 5 to about 500 mg, from about 10 to about 350 mg, or from about 50 to about 200 mg. The specific amount of the agent will depend on the specific agent used, the type of cancer-related disease or disorder being treated or managed, and the amount(s) of an immunomodulatory compound and any optional additional active agents concurrently administered to the patient.

5.4.1 Parenteral Dosage Forms

Parenteral dosage forms can be administered to patients by various routes including, but not limited to, subcutaneous, intravenous (including bolus injection), intramuscular, intraarterial, and the like. Parenteral administration typically bypasses an individual's natural defenses against contaminants, so these dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, emulsions, and the like.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, benzyl benzoate, and the like.

Compounds that increase the solubility of one or more of the active ingredients disclosed herein can also be incorporated into parenteral dosage forms. For example, cyclodextrin and its derivatives can be used to increase the solubility of an immunomodulatory compound and its derivatives. (See, e.g., U.S. Pat. No. 5,134,127, which is incorporated herein by reference).

5.4.2 Oral Dosage Forms

One or more of the components of the composition can be administered orally, if desired. Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms can be prepared by combining the active ingredients with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, coloring agents, and the like. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, disintegrating agents, and the like.

If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, lubricants, and the like. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, mixtures thereof, and the like.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, mixtures thereof, and the like. The binder or filler in pharmaceutical compositions is typically present in from about 50 to about 99 weight percent of the pharmaceutical composition or dosage form.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, mixtures thereof, and the like.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, mixtures thereof, and the like.

5.4.3 Delayed Release Dosage Forms

One or more of the active components of the combination composition can be administered by a delayed release means, if desired. Controlled release means or by delivery devices are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients. Also provided herein are single unit dosage forms suitable for oral administration such as, but not limited to, tablets, capsules, gelcaps, and caplets that are adapted for controlled-release.

Among the advantages of controlled-release formulations are the extended activity of the drug, reduced dosage frequency, and increased patient compliance. In addition, controlled-release formulations can be used to affect the time of onset of action or other characteristics, such as blood levels of the drug, and can thus affect the occurrence of side effects.

Most controlled-release formulations are designed to initially release an amount of the active ingredient that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.4.4 Topical and Mucosal Dosage Forms

In some embodiments, at least one of the components of the combination formulation can be administered topically or mucosally. Topical and mucosal dosage forms include, but are not limited to, sprays, aerosols, solutions, emulsions, suspensions, eye drops or other ophthalmic preparations, or other forms known to one of skill in the art. (See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985)). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms are well known to those skilled in the pharmaceutical arts, and depend on the particular tissue to which a given pharmaceutical composition or dosage form will be applied. Typical excipients include, but are not limited to, water, acetone, ethanol, ethylene glycol, propylene glycol, butane-1,3-diol, isopropyl myristate, isopropyl palmitate, mineral oil, mixtures thereof, and the like. The excipients can form solutions, emulsions or gels, which are non-toxic and pharmaceutically acceptable. Moisturizers or humectants can also be added to pharmaceutical compositions and dosage forms if desired. Examples of such additional ingredients are well known in the art. (See, e.g., Remington's Pharmaceutical Sciences, 16th and 18th eds., Mack Publishing, Easton Pa. (1980 & 1990)).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Various salts, hydrates or solvates of the active ingredients can be used to further adjust the properties of the resulting composition.

5.4.5 Kits

In some embodiments, a kit for treating cancer is provided. A typical kit comprises the combination of a dosage form of an immunomodulatory compound, or a pharmaceutically acceptable salt, solvate, stereoisomer, or prodrug thereof, a CD40 antibody, and/or CD40L. Kits can further comprise additional active and inactive ingredients.

Kits can further comprise devices that are used to administer the active ingredients. Examples of such devices include, but are not limited to, syringes, drip bags, patches, inhalers, and the like. Kits can also contain instruction sheets for use. The kits can be for single use, or can be designed for multiple dosage use.

Kits can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, benzyl benzoate, and the like.

The following examples are offered for illustrative purposes only, and are not intended to limit the scope of the present invention in any way.

### 6. EXAMPLES

### 6.1 Preparation of Cell Lines and Primary B-CLL Cells

L929 cells are derived from an immortalized mouse fibroblast cell line, and are routinely used in in-vitro assays. The L929 cells used herein were obtained from the American Type Culture Collection (ATCC). Raji cells were also obtained from ATCC. Namalwa cells were obtained from the German Resource Centre for Biological Materials DSMZ. B-CLL cells were obtained from the peripheral blood of patients enrolled in a clinical trial.

Namalwa, Raji, CD40L-transfected fibroblasts and L929 fibroblasts were cultured in RPMI medium (Gibco-Invitrogen) supplemented with 10% FBS. B-CLL cells were cultured in Iscove's medium (Gibco-Invitrogen) plus 15% FBS. Namalwa, Raji and B-CLL cells were cultured with compounds from 2 to 4 days. Fibroblasts were maintained in culture in monolayers. The day before the co-culture, the fibroblasts were treated with mitomycin C to stop proliferation and were then replated. Suspension cells were then added to the fibroblasts and the culture was incubated for 2 to 4 days.

### 6.2 Flow Cytometry

Flow cytometry was then used to determine cell numbers and the percentage of apoptotic cells using the FACSArray system (BD Biosciences). The cells were stained with 7-Amino-Actinomycin D (7-AAD) (BD Pharmingen) for 10 minutes at room temperature prior to flow cytometry analysis in order to identify non-viable cells.

### 6.3 RNA Purification and Microarray Analysis

Total RNA was isolated from Namalwa and B-CLL cells using RNAeasy (Qiagen). Gene expression analysis was performed using Affymetrix human U133A gene chips. Double-stranded cDNA was synthesized using 5 mg of total RNA. Biotin-labeled cRNA was synthesized using a MessageAmp aRNA kit (Ambion). Five mg of cRNA was fragmented and hybridized to each array. The above procedures were performed twice for each RNA sample to obtain replicate biotin-labeled probes.

### 6.4 Effect of Immunomodulatory Compounds on Normal Primary B Cells

Cells cultured in the presence or absence of 1,3-dioxo-2-(2,6-dioxopiperidin-3-yl)-4-aminoisoindoline or 1-oxo-2-(2,6-dioxopiperidin-3-yl)-4-aminoisoindoline were characterized using flow cytometry and gene array analysis. The addition of immunomodulatory compounds 1,3-dioxo-2-(2,6-dioxopiperidin-3-yl)-4-aminoisoindoline or 1-oxo-2-(2,6-dioxopiperidin-3-yl)-4-aminoisoindoline to a culture of normal primary B cells did not increase the level of p21 expression, when measured after 24 hours and 72 hours of culture. However, both 1,3-dioxo-2-(2,6-dioxopiperidin-3-yl)-4-aminoisoindoline or 1-oxo-2-(2,6-dioxopiperidin-3-yl)-4-aminoisoindoline administration resulted in a dose-dependent increase in viable normal B cells. These results demonstrated that immunomodulatory compounds protect B cells from apoptosis and suggest that the anti-proliferation effect of immunomodulatory compounds is specific to malignant cells.

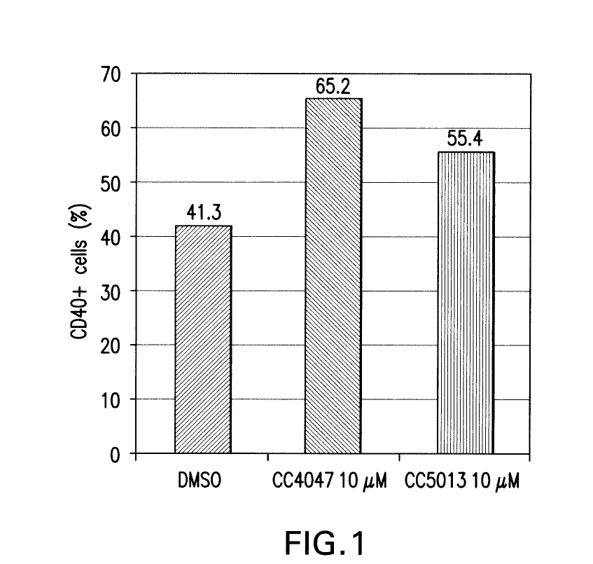

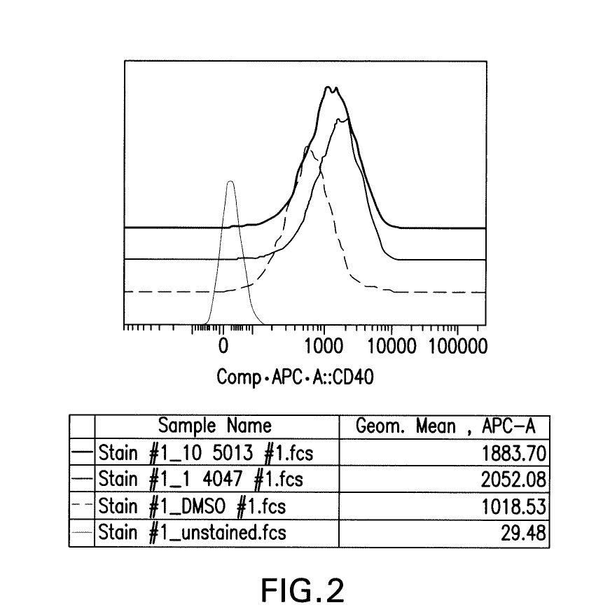

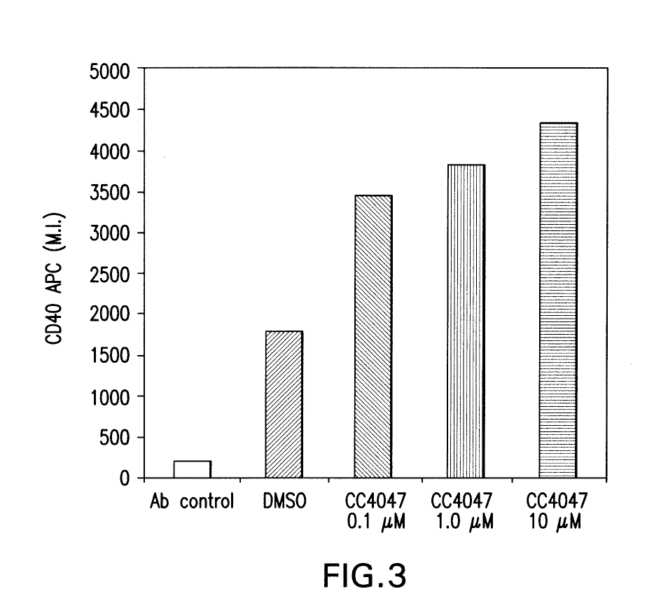

FIG. 1 demonstrates the upregulation of the B cell activation marker CD40 in Raji cells after administration of the immunomodulatory compounds 1,3-dioxo-2-(2,6-dioxopiperidin-3-yl)-4-aminoisoindoline or 1-oxo-2-(2,6-dioxopiperidin-3-yl)-4-aminoisoindoline. The upregulation of CD40 in CLL cells from a patient after four days of culture with an immunomodulatory compound is shown in FIG. 2. The upregulation effect exhibited a dose responsive manner, as illustrated in FIG. 3, when the mean intensity of anti-CD40 APC antibody on CD5⁺ CD 19⁺ CLL blasts isolated from a patient was measured. Further, the treatment of Namalwa and B-CLL with CD40L (in the form of CD40L transfected fibroblasts) was found to inhibit cell growth and/or augment cell apoptosis.

### 6.5 Immunomodulatory Compounds can Increase the Sensitivity to CD40 Ligation by CD40L

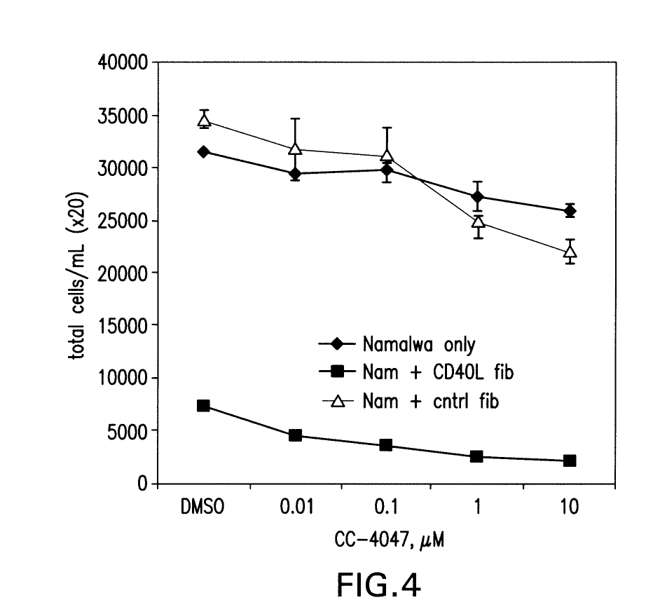

Pre-treatment of cells with immunomodulatory compounds 1,3-dioxo-2-(2,6-dioxopiperidin-3-yl)-4-aminoisoindoline and 1-oxo-2-(2,6-dioxopiperidin-3-yl)-4-aminoisoindoline increased the sensitivity of Namalwa cells to CD40 ligation by CD40L (in the form of CD40L transfected fibroblasts), as shown in FIG. 4.

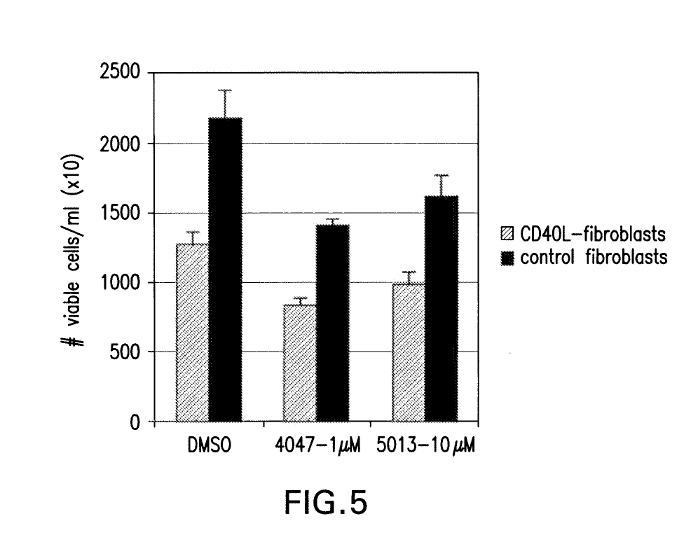

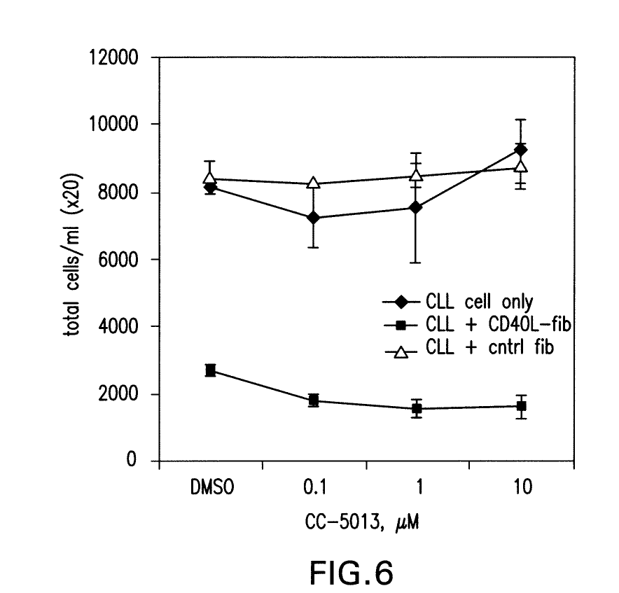

Pre-treatment with immunomodulatory compounds also augmented the sensitivity of Namalwa cells and CLL cells to CD40 ligation by CD40L. B-CLL cells taken from a relapsed patient at baseline were pre-treated with either 1,3-dioxo-2-(2,6-dioxopiperidin-3-yl)-4-aminoisoindoline or 1-oxo-2-(2,6-dioxopiperidin-3-yl)-4-aminoisoindoline for 48 hours. The cells were then cocultured with either CD40L-transfected fibroblasts or control fibroblasts for another 48 hours. The percentage of viable cells per ml was then measured (FIG. 5). Cells from another patient were pretreated with 1-oxo-2-(2,6-dioxopiperidin-3-yl)-4-aminoisoindoline for 24 hours and then cocultured with either CD40L-transfected fibroblasts or control fibroblasts for an additional 48 hours. The results, measured in total cells/ml, show that addition of 1-oxo-2-(2,6-dioxopiperidin-3-yl)-4-aminoisoindoline increases the sensitivity to the CD40L treatment (FIG. 6).

### 6.6 Immunomodulatory Compounds Upregulate CD40 in B-CLL Cells and Upregulate CD40L in T Cells

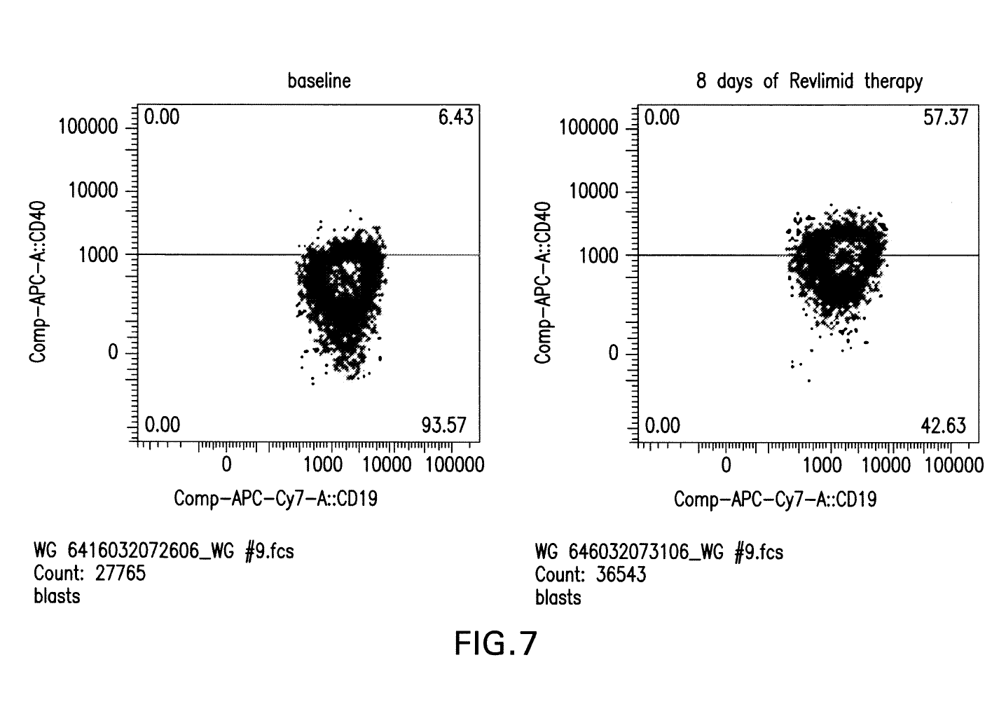

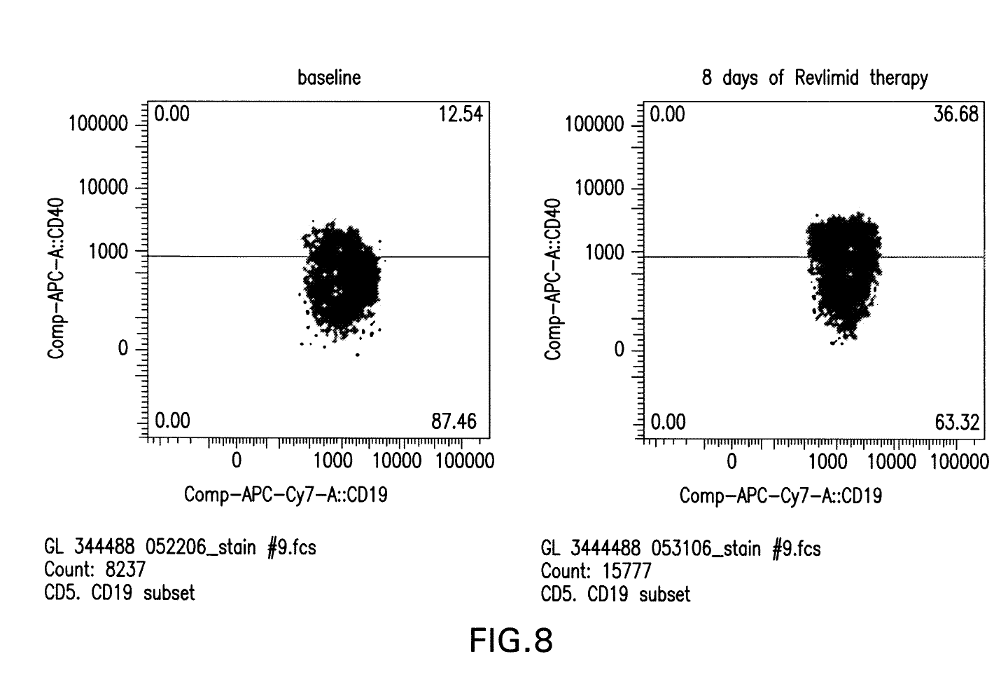

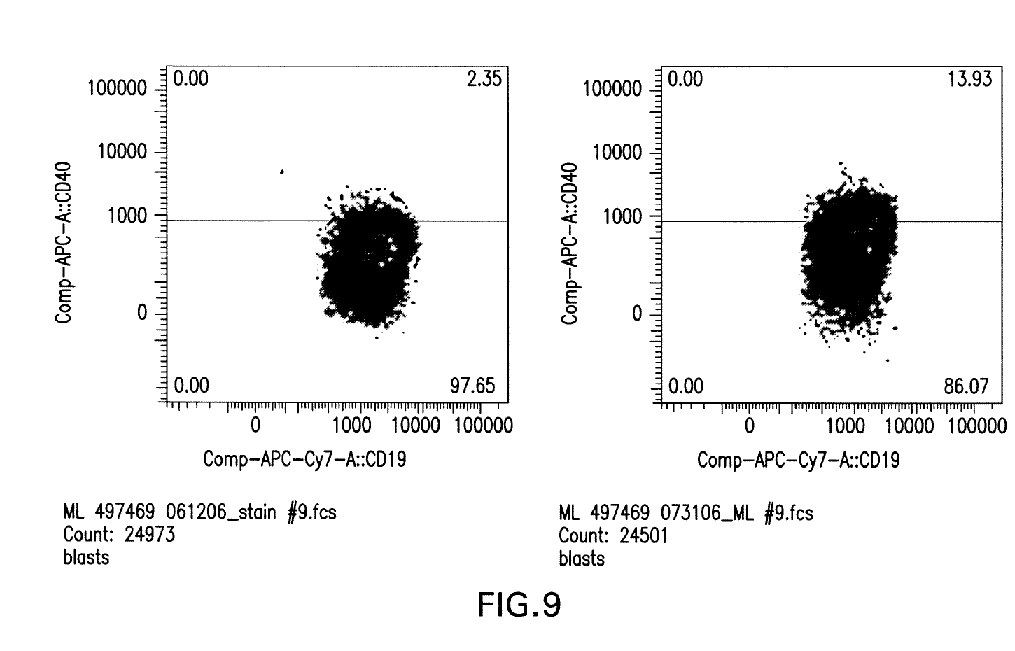

Administration of the immunomodulatory compound 1-oxo-2-(2,6-dioxopiperidin-3-yl)-4-aminoisoindoline results in an upregulation of CD40 in B-CLL cells of patients undergoing 1-oxo-2-(2,6-dioxopiperidin-3-yl)-4-aminoisoindoline therapy. The level of CD40 expression on CD19⁺CD5⁺ cells isolated from individual patients after 8 days of 1-oxo-2-(2,6-dioxopiperidin-3-yl)-4-aminoisoindoline treatment was measured using flow cytometry. The two-dimensional dot plot analysis from three different patients is shown in FIGS. 7, 8 and 9.

Certain immunomodulatory compounds were previously found to upregulate CD40L in T cells (Corral, L. G et al. Jour. Immunol., 163:380-6 (1999), which is incorporated by reference herein in its entirety). Without being limited by a particular theory, the two complementary effects—the upregulation of CD40 on malignant cells and the upregulation of CD40L on T cells) combined may be beneficial to the recognition of the malignant cells by T lymphocytes and the triggering or augmentation of antitumor activity.

### 6.7 Combination Treatment of an Immunomodulatory Compound and CD40L to Treat Cancer

Soluble CD40L is obtained by recombinant methods and is purified. A patient with a B cell related cancer is identified. The patient is administered 1-oxo-2-(2,6-dioxopiperidin-3-yl)-4-aminoisoindoline at a dose of 10 mg/kg twice per week. After 2 weeks, the patient is additionally administered purified, soluble CD40L in a weekly dose of 5 mg/kg by intravenous injection. After 1 week, a blood sample is taken, and the effect of the compound administration is examined. By use of this method, the level of B cell related cancer decreases.

### 6.8 Combination Treatment of an Immunomodulatory Compound and an Anti-CD40 Antibody to Treat Cancer

An anti-CD40 antibody is obtained commercially. A patient with a B cell related cancer is identified. The patient is administered an oral dose of 1,3-dioxo-2-(2,6-dioxopiperidin-3-yl)-4-aminoisoindoline at a dose of 80 mg/kg once a week for 6 months. The patient also receives a once per week injection (50 mg/kg) of the anti-CD40 antibody for 6 months. Monthly blood samples are taken, and the effect of the compound administration is examined. By use of this method, the level of B cell related cancer decreases and patient health improves.

All of the references cited herein are incorporated by reference in their entirety. While the invention has been described with respect to the particular embodiments, it will be apparent to those skilled in the art that various changes and modifications may be made without departing from the spirit and scope of the invention as recited by the appended claims.

The embodiments provided herein described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed:

1. A method of treating cancer in a patient, comprising administering to the patient (i) an effective amount of CD40L, derivative, or fragment thereof, and (ii) an effective amount of an immunomodulatory compound, wherein the immunomodulatory compound is a compound of formula I, II, III, IV, V, VI, VII, VIII, IX, X, XI, XII, XIII, XIV, XV, XVI, XVII, XVIII or a pharmaceutically acceptable salt, solvate, or stereoisomer thereof: 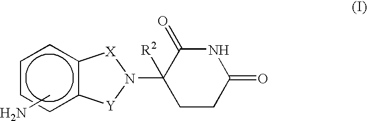
wherein:
one of X and Y is C═O, the other of X and Y is C═O or CH₂;
R² is hydrogen or lower alkyl; 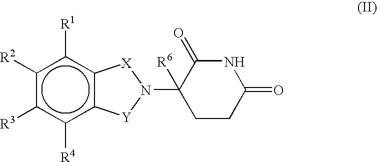
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms, or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;
provided that R⁶ is other than hydrogen if X and Y are C═O and (i) each of R¹, R², R³, and R⁴ is fluoro or (ii) one of R¹, R², R³, or R⁴ is amino; 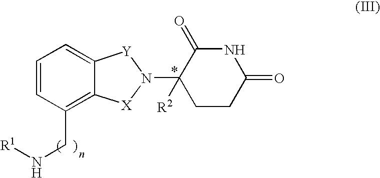
wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl C₁-C₆)heterocycloalkyl, (C₀-C₄)alkyl C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R³, C(S)NR³R³ or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* is a chiral-carbon center; 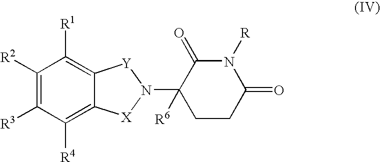
wherein:
one of X and Y is C═O and the other is CH₂ or C═O;
R is H or CH₂OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;
R¹⁰ is hydrogen, alkyl of to 8 carbon atoms, or phenyl; and
* represents a chiral-carbon center; 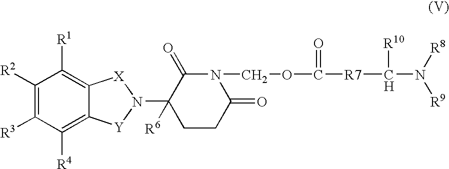
wherein:
one of X and Y is C═O and the other of X and Y is C═O or CH₂;
(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzo, chloro, or fluoro;
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and
R¹⁰ is hydrogen, alkyl of to 8 carbon atoms, or phenyl; 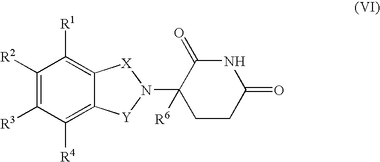
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and
R⁶ is hydrogen, alkyl of 1 to 8 carbon atoms, benzo, chloro, or fluoro; 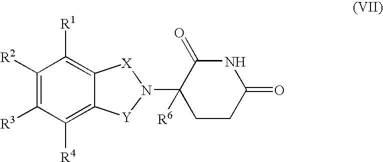
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and
R⁶ is alkyl of 1 to 8 carbon atoms, benzo, chloro, or fluoro; 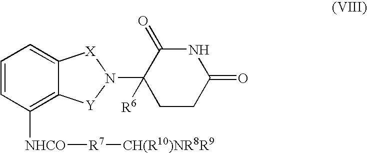
wherein:
one of X and Y is C═O and the other of X and Y is C═O or CH₂;
R⁶ is hydrogen, alkyl of 1 to 8 carbon atoms, benzyl, chloro, or fluoro;
R⁷ is m-phenylene, p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and
R¹⁰ is hydrogen, alkyl of 1 to 8 carbon atoms, or phenyl; 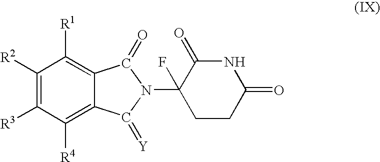
wherein:
Y is oxygen or H² and
each of R¹, R², R³, and R⁴, independently of the others, is hydrogen, halo, alkyl of 1 to 4 carbon atoms, alkoxy of 1 to 4 carbon atoms, or amino; 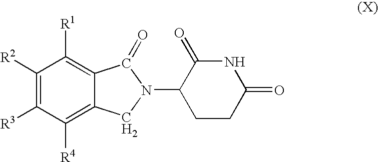
wherein:
each of R¹, R², R³, and R⁴, independently of the others, is halo, alkyl of 1 to 4 carbon atoms, or alkoxy of 1 to 4 carbon atoms; 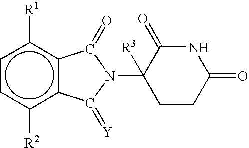
wherein:
Y is Oxygen or H₂,
a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and
R³ is hydrogen, alkyl, or benzyl; 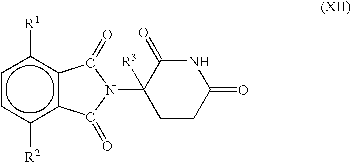
wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl; 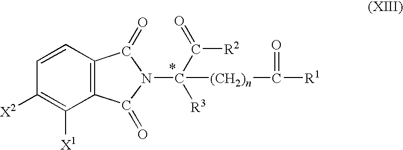
wherein:
when n is not zero and R¹ is not the same as R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is hydrogen, alkyl of one to six carbons, halo, or haloalkyl;
Z is hydrogen, aryl, alkyl of one to six carbons, formyl, or acyl of one to six carbons; and
n has a value of 0, 1, or 2;
provided that if X¹ is amino, and n is 1 or 2, then R¹ and R² are not both hydroxy; 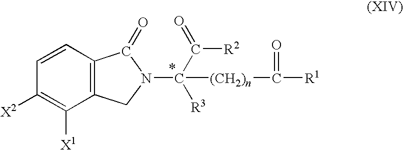
wherein:
when n is not zero and R¹ is not R², C* is a center of chirality;
one of X and X² is amino, nitro, alkyl of one to six carbons, or NH-Z, and the other of X or X² is hydrogen;
each of R¹ and R² independent of the other, is hydroxy or NH-Z; R³ is alkyl of one to six carbons, halo, or hydrogen;
Z is hydrogen, aryl or an alkyl or acyl of one to six carbons; and
n has a value of 0, 1, or 2; 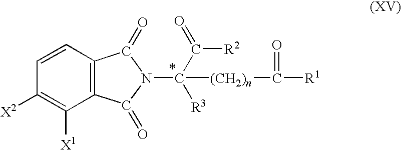
wherein:
when n is not zero and R¹ is not R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is alkyl of one to six carbons, halo, or hydrogen;
Z is hydrogen, aryl, or an alkyl or acyl of one to six carbons; and
n has a value of 0, 1, or 2; 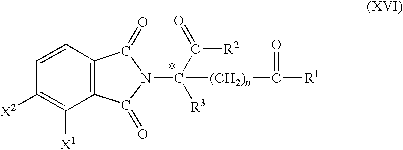
wherein:
one of X¹ and X² is nitro, or NH-Z, and the other of X¹ or X² is hydrogen;
each of R¹ and R², independent of the other, is hydroxy or NH-7;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality; 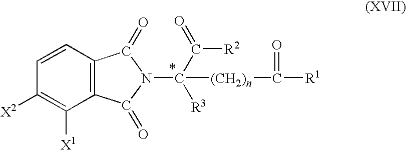
wherein:
one of X¹ and X² is alkyl of one to six carbons;
each of R¹ and R², independent of the other, is hydroxy or NH-Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality; 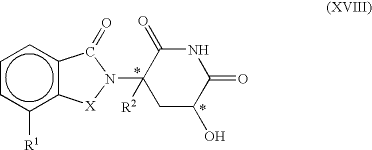
wherein:
the * carbons are centers of chirality;
X is —C(O)— or —CH₂—;
R¹ is alkyl of 1 to 8 carbon atoms or —NHR³;
R² is hydrogen, alkyl of 1 to 8 carbon atoms, or halogen; and
R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴, wherein
R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

2. The method of claim 1, wherein the immunomodulatory compound is administered before, concurrently with, or after the administration of the CD40L, derivative, or fragment thereof.

3. The method of claim 2, wherein the immunomodulatory compound is administered from about 1, 6, 12, or 24 hours to about 2 days, 4 days, 1 week, or about 2 weeks before the administration of the CD40L, derivative, or fragment thereof.

4. The method of claim 1, wherein the cancer is solid or hematological cancer.

5. The method of claim 1, wherein the cancer is a leukemia, lymphoma or myeloma.

6. The method of claim 5, wherein the leukemia is chronic lymphocytic leukemia (CLL), acute lymphoblastic leukemia (ALL), prolymphocytic leukemia (PLL), hairy cell leukemia, or small lymphocytic leukemia (SLL).

7. The method of claim 5, wherein the lymphoma is selected from the group consisting of Mantle cell lymphoma, splenic lymphoma, hodgkin's lymphoma, mucosal associated lymphoid tissue lymphoma, diffuse small lymphocytic lymphoma, follicular lymphoma, mocytoid B cell lymphoma, Burkitt's lymphoma, AIDS-related lymphoma, diffuse large B-cell lymphoma, lymphomatoid granulomatosis, intravascular lymphomatosis, intravascular lymphoma, cutaneous B-cell lymphoma, and non-hodgkins lymphoma.

8. The method of claim 1, wherein the cancer is multiple myeloma.

9. The method of claim 1, wherein the immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

10. The method of claim 1, wherein the immunomodulatory compound is administered in an amount between about 2 to about 100 mg/kg.

11. The method of claim 1, wherein the immunomodulatory compound is administered orally, parenterally, or topically.

12. The method of claim 1, further comprising administering an effective amount of an anti-CD40 antibody or a fragment thereof.

13. A method of treating leukemia or lymphoma in a patient, comprising administering to the patient: (i) an effective amount of an anti-CD40 antibody or a fragment thereof, and (ii) an effective amount of an immunomodulatory compound, wherein the immunomodulatory compound is a compound of formula I, II, III, IV, V, VI, VII, VIII, IX, X, XI, XII, XIII, XIV, XV, XVI, XVII, XVIII or a pharmaceutically acceptable salt, solvate, or stereoisomer thereof: 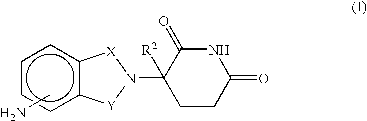
wherein:
one of X and Y is C═O, the other of X and Y is C═O or CH₂;
R² is hydrogen or lower alkyl; 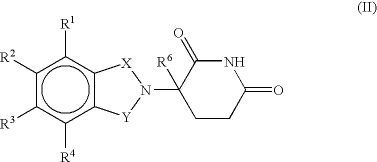
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms, or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;
provided that R⁶ is other than hydrogen if X and Y are C═O and (i) each of R¹, R², R³, and R⁴ is fluoro or (ii) one of R¹, R², R³, or R⁴ is amino; 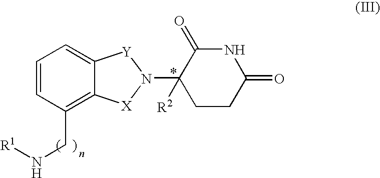
wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl C₁-C₆)heterocycloalkyl, (C₀-C₄)alkyl C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R³, C(S)NR³R³ or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* is a chiral-carbon center; 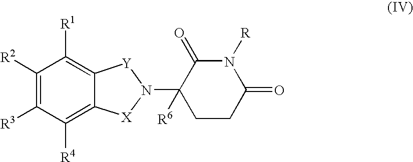
wherein:
one of X and Y is C═O and the other is CH₂ or C═O;
R is H or CH₂OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X¹ is —O—, —S—, or —NH—;
R¹⁰ is hydrogen, alkyl of to 8 carbon atoms, or phenyl; and
* represents a chiral-carbon center; 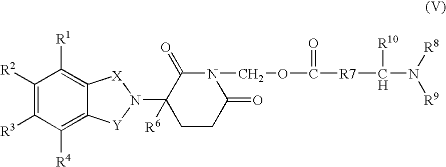
wherein:
one of X and Y is C═O and the other of X and Y is C═O or CH₂;
(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzo, chloro, or fluoro;
R⁷ is m-phenylene or p-phenylene or —(CⁿH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and
R¹⁰ is hydrogen, alkyl of to 8 carbon atoms, or phenyl; 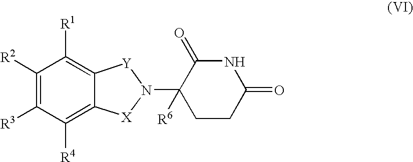
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and
R⁶ is hydrogen, alkyl of 1 to 8 carbon atoms, benzo, chloro, or fluoro; 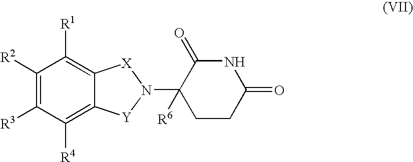
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and
R⁶ is alkyl of 1 to 8 carbon atoms, benzo, chloro, or fluoro; 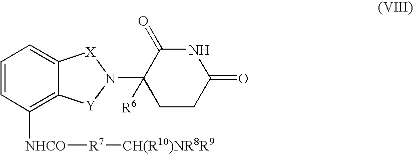
wherein:
one of X and Y is C═O and the other of X and Y is C═O or CH₂;
R⁶ is hydrogen, alkyl of 1 to 8 carbon atoms, benzyl, chloro, or fluoro;
R⁷ is m-phenylene, p-phenylene or —(CⁿH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and
R¹⁰ is hydrogen, alkyl of 1 to 8 carbon atoms, or phenyl; 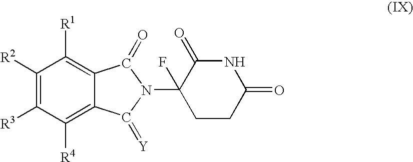
wherein:
Y is oxygen or H² and
each of R¹, R², R³, and R⁴, independently of the others, is hydrogen, halo, alkyl of 1 to 4 carbon atoms, alkoxy of 1 to 4 carbon atoms, or amino; 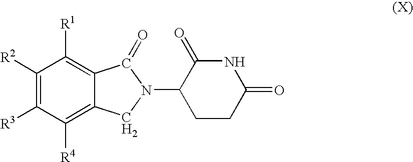
wherein:
each of R¹, R², R³, and R⁴, independently of the others, is halo, alkyl of 1 to 4 carbon atoms, or alkoxy of 1 to 4 carbon atoms; 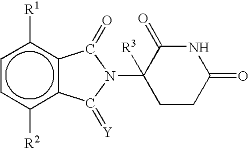
wherein:
Y is Oxygen or H₂,
a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and
R³ is hydrogen, alkyl, or benzyl; 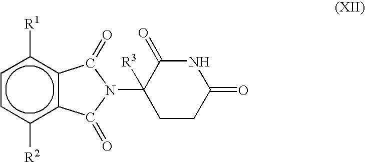
wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl; 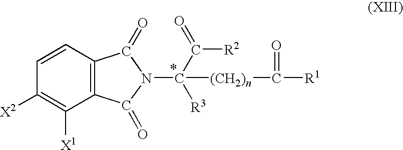
wherein:
when n is not zero and R¹ is not the same as R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is hydrogen, alkyl of one to six carbons, halo, or haloalkyl;
Z is hydrogen, aryl, alkyl of one to six carbons, formyl, or acyl of one to six carbons; and
n has a value of 0, 1, or 2;
provided that if X¹ is amino, and n is 1 or 2, then R¹ and R² are not both hydroxy; 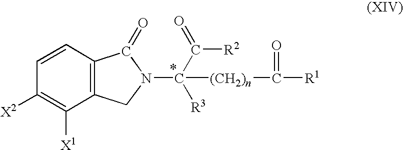
wherein:
when n is not zero and R¹ is not R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is alkyl of one to six carbons, halo, or hydrogen;
Z is hydrogen, aryl or an alkyl or acyl of one to six carbons; and
n has a value of 0, 1, or 2; 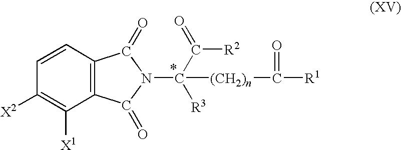
wherein:
when n is not zero and R¹ is not R², C* is a center of chirality;
one of X¹ and X² is amino, nitro, alkyl of one to six carbons, or NH-Z, and the other of X¹ or X² is hydrogen;
each of R¹ and R² independent of the other, is hydroxy or NH-Z; R³ is alkyl of one to six carbons, halo, or hydrogen;
Z is hydrogen, aryl, or an alkyl or acyl of one to six carbons; and
n has a value of 0, 1, or 2; 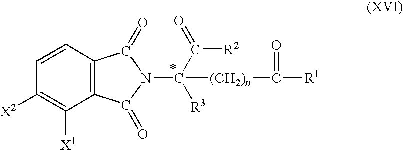
wherein:
one of X¹ and X² is nitro, or NH-Z, and the other of X¹ or X² is hydrogen;
each of R¹ and R², independent of the other, is hydroxy or NH-Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality; 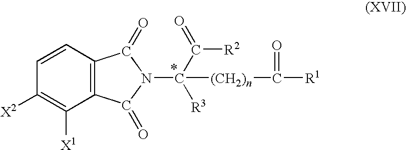
wherein:
one of X¹ and X² is alkyl of one to six carbons;
each of R¹ and R², independent of the other, is hydroxy or NH-Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality; 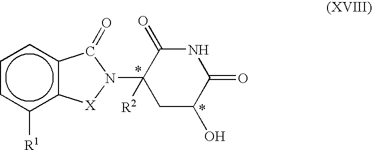
wherein:
the carbons are centers of chirality;
X is —C(O)— or —CH₂—;
R¹ is alkyl of 1 to 8 carbon atoms or —NHR³;
R² is hydrogen, alkyl of 1 to 8 carbon atoms, or halogen; and
R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴, wherein
R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

14. The method of claim 13, wherein the immunomodulatory compound is administered before, concurrently with, or after administration of the anti-CD40 antibody or fragment thereof.

15. The method of claim 14, wherein the immunomodulatory compound is administered from about 1, 6, 12, or 24 hours to about 2 days, 4 days, 1 week, or about 2 weeks before administration of the anti-CD40 antibody or fragment thereof.

16. The method of claim 13, wherein the leukemia is chronic lymphocytic leukemia (CLL), acute lymphoblastic leukemia (ALL), prolymphocytic leukemia (PLL), hairy cell leukemia, or small lymphocytic leukemia (SLL).

17. The method of claim 13, wherein the lymphoma is selected from the group consisting of Mantle cell lymphoma, splenic lymphoma, hodgkin's lymphoma, mucosal associated lymphoid tissue lymphoma, diffuse small lymphocytic lymphoma, follicular lymphoma, mocytoid B cell lymphoma, Burkitt's lymphoma, AIDS-related lymphoma, diffuse large B-cell lymphoma, lymphomatoid granulomatosis, intravascular lymphomatosis, intravascular lymphoma, cutaneous B-cell lymphoma, and non-hodgkins lymphoma.

18. The method of claim 13, wherein said immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

19. The method of claim 13, wherein said immunomodulatory compound is administered in an amount of between about 2 to about 100 mg/kg.

20. The method of claim 13, wherein said immunomodulatory compound is administered orally, parenterally, or topically.
